(19) United States
(12) Patent Application Publication
(10) Pub. No.: US 2023/0172664 A1
(43) Pub. Date: Jun. 8, 2023

(54) METHODS AND KITS FOR OPTIMIZATION OF NEUROSURGICAL INTERVENTION SITE

(71) Applicants: Abdullah ZAHID, Minneapolis, MN (US); The United States Government as represented by the Department of Veterans Affairs, Washington, DC (US); Hennepin Healthcare Systems, Inc., Minneapolis, MN (US)

(72) Inventors: Uzma Samadani, Wayzata, MN (US); Abdullah Bin Zahid, Norman, OK (US)

(21) Appl. No.: 17/048,938

(22) PCT Filed: Apr. 19, 2019
(86) PCT No.: PCT/US2019/028333

§ 371 Date: Oct. 19, 2020

Related U.S. Application Data
(60) Provisional application No. 62/660,717, filed on Apr. 20, 2018.

(51) Int. Cl. A61B 34/10; G16H 20/40; G06T 7/11; G06T 7/00

(52) U.S. Cl. CPC A61B 34/10; G16H 20/40; G06T 7/11; G06T 7/0016; A61B 2034/107; A61B 2034/105; A61B 2090/367

(57) ABSTRACT

Methods and systems for aiding in placement of surgical intervention sites for treating lesions for optimization of surgical outcomes are provided. Also disclosed are computer-aided software packages for identifying the optimal placement of a surgical intervention on a patient in need thereof. The packages, systems, and methods for optimizing placement provided herein help to achieve superior results (e.g. increased drainage), decrease hospitalization time, reduce recurrence of lesions and need for drainage, and improve cognitive outcomes for patients.

### CROSS REFERENCE TO RELATED APPLICATIONS

This is a U.S. National Phase application of International Application No. PCT/US2019/028333, filed Apr. 19, 2019, which claims priority to and the benefit of the filing date of U.S. Provisional Application No. 62/660,717, filed Apr. 20, 2018, which are incorporated herein by reference in their entireties.

### FIELD

The disclosed invention relates to methods for optimizing outcomes for neurosurgical interventions.

### BACKGROUND

Chronic subdural hematoma (cSDH) has become increasingly prevalent in the aging civilian and veteran population and is projected to become the most common indication for an adult cranial procedure in the United States by 2030¹. It is tenfold more common among Veterans Administration patients than civilians¹ and has high mortality, with 32% of afflicted subjects between ages 65 and 96 dying within one year of diagnosis². cSDH has a high recurrence rate³⁻⁶, and patients often require prolonged hospitalization and rehabilitation^{7,8}.

Patients treated for cSDH are at risk for intracerebral hemorrhage, seizures, exacerbation of comorbidities associated with the interruption of anticoagulant therapy, and other complications associated with hospitalization of the elderly. Up to 20% of patients have poor neurologic outcomes resulting in significant disability⁹⁻¹². One-year mortality among elderly patients treated with a drainage intervention is 30% to 32%². The mean survival of post-cSDH patients is 4.4 to 4.7 years, which is significantly shorter (hazard ratio of 1.94, p<0.0002) than the mean peer survival of 6.0 years computed from actuarial life tables². The mortality rate for relatively younger cSDH patients, aged 55 to 64 years, is 17 times that of the age-matched general population rate⁹⁻¹². The median length of hospital stay for a cSDH is 8 days, which is higher than the median length of stay for age-matched patients undergoing brain tumor resection performed by the same neurological service⁹.

cSDH has traditionally been treated by surgical drainage via craniotomy or burr hole craniostomy in the operating room, or more recently by twist drill craniostomy (TDC) at the patient bedside. The purpose of drainage for cSDH is not only to relieve immediate mass effect on the brain, but also to remove toxic blood break-down products. Iron toxicity is well-established as a potential effector of cognitive outcome¹³⁻¹⁶. Increased extent of drainage of cSDH correlates with improved clinical outcomes such as increased survival¹⁷, reduced recurrence^{18,19} and better functional outcome²⁰.

The current standard of care for drainage of cSDH is that the surgeon approximates optimal burr hole or craniostomy placement based on viewing of a series of two-dimensional computed tomography (CT) images. What is needed is an improved method for placement of drainage sites to optimize drainage.

### SUMMARY

Described herein, in various aspects, is a method for optimizing placement of a surgical intervention site for a surgical intervention in a human or animal subject. The method can comprise imaging a lesion in the subject, segmenting the lesion, identifying a center of the lesion along a z-axis, identifying an anterior pole of the lesion along an anteroposterior axis, and displaying a location for the surgical intervention in a three dimensional representation of at least a portion of the subject.

Imaging the lesion in the subject can comprise using an imaging method selected from radiography, computed tomography, medical resonance imaging, or ultrasound.

At least a portion of the method can be performed by a processor executing a computer program.

The processor, when executing the computer program, can apply a machine learning algorithm for determining the location for the surgical intervention.

The processor, when executing the computer program, can provide, on a visual output, an interface for displaying and coregistering a pre-procedure image and a post-procedure image.

Coregistering can be performed using an intensity based coregistration, wherein the pre-procedure image is a fixed target.

The method can further comprise performing a surgical intervention, wherein the surgical intervention is an incision, a drainage, a drilling, or a combination thereof.

The surgical intervention site can be a drill site.

The lesion can be a collection or accumulation of fluid within a brain, a spine, a subdural space, or an epidural space of the subject.

The lesion can be a subdural hematoma, wherein performing the surgical intervention comprises draining more than about 70% of a volume of the subdural hematoma.

Performing the surgical intervention can comprise draining more than about 80% of the volume of the subdural hematoma.

The location for the surgical intervention site can be at the anterior pole of the lesion along the anteroposterior axis and at the center of the lesion along the z-axis.

A method for assessing the volumetric distribution of a brain lesion can comprise imaging the brain lesion, using a processor, performing segmentation analysis of an image of the brain lesion to determine an anterior pole of the brain lesion along an anteroposterior axis and a center of the brain lesion along a z-axis; using the processor, creating a model of the brain lesion including the anterior pole of the brain lesion and the center of the brain lesion along the z-axis; and using the processor, identifying the anterior pole of the brain lesion along the anteroposterior axis and the center of the brain lesion along the z-axis as a location for a surgical approach to treat the brain lesion.

The brain lesion can be a collection or accumulation of fluid within a brain, a spine, a subdural space, or an epidural space.

The surgical approach can be a twist drill craniostomy.

The surgical approach can comprise placing a drain for a subdural hematoma.

Identifying the anterior pole of the brain lesion and the center of the brain lesion along the z-axis as the location for the surgical approach can comprise locating a treatment site for a treatment, wherein the treatment is one of a surgical incision, a cranial drill site, a craniostomy location, a craniotomy location, and a craniectomy location.

The segmentation analysis can include an analysis of images to distinguish between brain tissue and non-brain space.

The non-brain space can be an intracranial space containing one or more of cerebrospinal fluid, air, blood, a tumor, an abscess, a nodule, and an inflammatory lesion.

The segmentation analysis can include an analysis of one or more of a density of the brain lesion, volume of the brain lesion, area of distribution of the brain lesion, and a gravitational force acting upon the brain lesion.

Identifying the anterior pole of the brain lesion and the center of the brain lesion along the z-axis as the location for the surgical approach can comprise using the processor to analyze and compare pre-procedure and post-procedure imaging from patients who have undergone the treatment.

The location for the surgical approach can be at (or approximately at) an anterior pole of the brain lesion along an anteroposterior axis and at (or approximately at) a center of the brain lesion along a z-axis.

A method for assessing the volumetric distribution of a brain lesion can comprise imaging the brain lesion, using segmentation analysis to analyze the brain lesion, creating a model of the brain lesion, and identifying a location for a surgical approach to treat the brain lesion.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

These and other features of the preferred embodiments of the invention will become more apparent in the detailed description in which reference is made to the appended drawings wherein:

FIG. 1 shows a user interface of software used to perform manual segmentation described herein.

FIG. 2 illustrates an image of a typical coregistration result. Lighter areas show where the pre- and post-procedure scans' intensities are equal to each other, and darker areas show where the pre- and post-procedure scans' intensities differ. Moreover, the scans can be color-coded to illustrate the areas that are more intense in pre-procedure and the areas that are more intense in post-procedure scans.

FIG. 3 illustrates a plot of residual hematoma expressed as a percent of initial hematoma volume versus the distance of twist drill craniostomy (TDC) drain from the hematoma's centroid in millimeters. A line of best fit and 95% confidence intervals of coefficients are shown. The size of the individual dots is proportional to the size of the SDH prior to the drainage. As illustrated, the drain distance from the centroid does not correlate with the decrease in the residual hematoma (R=0.014, p=0.947).

FIG. 4 illustrates a plot of residual hematoma expressed as a percent of initial hematoma volume versus the placement of twist drill craniostomy along anteroposterior axis expressed as a percent, with zero being at the very anterior pole of the hematoma and 100% being at the very posterior pole of the hematoma. The sizes of the dots correlate with the size of the respective hematoma prior to the drainage, and the color indicates the age of the subject at the time of drainage. The line of best fit and 95% confidence intervals for the coefficients are shown. A strong correlation is shown between the anteriorly placed drains leading to lower amount of residual hematoma (R=0.566, p=0.003) holds for hematomas of all sizes and subjects of all ages equally.

FIG. 5 illustrates a plot of residual hematoma expressed as a percent of initial hematoma volume versus the placement of twist drill craniostomy along craniocaudal axis expressed as a percent, with zero being at the caudal most end of the hematoma and 100% being at the top end of the hematoma, with the sizes of the dots indicating the size of the respective hematoma prior to drainage. The line of best fit and 95% confidence intervals are shown. The placement along craniocaudal axis did not appear to be associated with the decreased residual hematoma (R=0.132, p=0.522).

FIG. 6 illustrates a plot of the data from FIG. 5, but as square root of the distance from the center of hematoma (2). It can be seen that the drains placed closer to the center of the hematoma (lower Y-values) had lower residual hematoma volumes (lower x-values).

FIG. 7 illustrates a plot of the data of FIG. 5 with both craniocaudal and anteroposterior axes combined, according to a model listed in Table 6 and described herein. The model could explain 71.2% of the drainage of the hematoma.

FIG. 8 illustrates a plot of the data from FIG. 7 with anteroposterior axes along y-axis and craniocaudal axis along x-axis, with individual data points sized according the pre-drainage hematoma and colored according to the percentage of the residual hematoma (also written as labels). The diagonal lines reflect the individual percentiles as predicted the model, for example, if drain is placed below the 2^{nd} line in the lower left-hand corner, 20% or less residual is expected. In the direction toward the upper right corner, the amount of residual hematoma increases.

FIG. 9 shows a perspective view of a 3D display of the subject's head and the area at which the model predicts 80% or more drainage in this particular subject for his particular chronic subdural hematoma.

FIG. 10 is a computing device for performing aspects of the methods disclosed herein.

FIG. 11 illustrates a schematic of a patient having a lesion and the area at which the model predicts 80% or more drainage in this particular subject for his particular chronic subdural hematoma.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, use of the term “a scan” can refer to one or more of such scans, and so forth.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “approximately” one particular value, and/or to “approximately” another particular value. When such a range is expressed, another aspect includes from the one particular value and/or to the other particular value. Similarly, when values are expressed as approximations, by use of the antecedent “approximately,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “approximately,” it is contemplated that values within up to 15%, up to 10%, up to 5%, or up to 1% (above or below) of the particularly stated value can be included within the scope of those aspects.

The word “or” as used herein means any one member of a particular list and also includes any combination of members of that list.

While operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

It should be understood that, as used herein, the “z-axis” refers to the vertical axis with respect to the orientation of the subject when standing. This corresponds to the craniocaudal axis for humans, although it should be understood that for various animal subjects, the z-axis could correspond to a different axis. Accordingly, although the z-axis and craniocaudal axis are used interchangeably herein, it should be understood that the z-axis refers to the vertical axis with respect to the orientation of the subject when the subject is standing.

Decisions about surgical incision and drill site location are generally made after review of a subject's history, physical examination of the subject, and medical imaging. Anatomical landmarks and neuronavigation systems are useful for optimizing surgical incision sites. Machine learning techniques have not historically been used to optimize surgical incision and drill sites.

Analysis of the medical images of multiple subjects obtained before and after surgery can be used to create a machine learning algorithm that optimizes surgical incision and drill site placement for any one subject. Such analyses can be performed for many different neurosurgical procedures for a variety of neuropathologies including trauma, degenerative disease, cancer, inflammatory pathologies, hydrocephalus, dementia, pathologies of the cerebrospinal fluid and its absorption, and others.

One potential indication for surgical site optimization using machine learning is drainage of fluid such as blood, its byproducts or cerebrospinal fluid on the surface of the brain, above or below the dura.

Traditional methods of subdural hematoma drainage often results in residual or recurrent hemorrhage. It is commonly assumed that placing a twist drill craniostomy (TDC) drain at the thickest or most central site of a subdural hematoma is associated with the best drainage. However, there has never been data to support this hypothesis. Therefore, new methods are provided to improve and optimize surgical sites in order to provide better options for surgeons and improved outcomes for patients (both human and non-human subjects).

Subjects who underwent TDC placement were retrospectively studied. Pre- and post-procedure scans were analyzed to measure the quantity of subdural hematoma. These scans were coregistered, and the TDC drain location was projected onto the pre-drainage scan. The distance from the drain location to a centroid based location was then calculated, as was the drain's location along the craniocaudal and anteroposterior axes.

Coregistration or co-registration may refer to a process for transforming data from two images into one coordinate system or image. As disclosed herein, data sets may be from the same subject taken at different times, for example medical images obtained before and after a surgical procedure. Coregistration can allow a clinician to compare, Integrate, and analyze the data obtained from the different data sets to easily view the changes and differences.

In one experiment, a total of 26 patients, mean age 76.75±10.52 years were studied. The average pre-procedure hematoma volume was calculated to be 131.64±52.18 ml, while average post procedure hematoma was 75.36±34.20 ml.

It was found that anterior placement and central placement of drains can be associated with significantly enhanced drainage. As described below, anterior placement correlates with decreased residual hematoma volume (Pearson correlation=0.566, p=0.003), and central placement along the craniocaudal axis (Pearson correlation=0.502, p=0.009). On the other hand, and contrary to existing practice, placing the drains closer to centroid of the hematoma is not associated with decreased residual hematoma volume. Likewise, patient age and smaller size of the hematoma also does not associate with decreased residual hematoma volume. It can be shown that combining both parameters, anterior and central placement, in selection of the drain site accounted for drainage of 71% of the initial hematoma.

TDC drains placed centrally and anteriorly can correlate with better drainage results and, thus, patient outcomes. Surprisingly, it can be shown that placing the drain site closer to the centroid of hematoma does not lead to better drainage results. In various embodiments, the disclosed method and system can be useful in identifying an area for placement of the intervention where 80% or more of the lesion may be removed. For example, referring to FIG. 9, in the case where a hematoma should be drained, the highlighted area 202 indicates a drain placement location at which 80% or more drainage is expected.

Described herein is a computer implemented program, application, and system for allowing clinicians to visualize optimal placement (for example rendering an optimal or golden area) on a patient's CT scan (or displayed on the patient herself).

Anecdotally, it is suggested that TDC should be performed at the site of maximum thickness of cSDH, but no studies to date have shown this to be the case in an actual patient population.

Placement of drain sites were analyzed retrospectively in subdural hematomas to correlate site placement with drainage results. Placement of drain sites along both the craniocaudal and anteroposterior axes were also analyzed.

Methods and systems disclosed herein can aid in placement of a surgical intervention site for treating a lesion, for optimization of surgical outcomes. In an embodiment, the lesion is a subdural hematoma. In an embodiment, the intervention is drainage of the subdural hematoma. In an embodiment, the surgical intervention is a drain placement and the surgical outcome is drainage. Also disclosed herein are factors for incorporation into algorithms of computer software packages for identifying the optimal placement of a surgical intervention on a patient in need thereof.

The disclosed packages, systems, and methods for optimizing placement can help to improve results (e.g. drainage), shorten hospitalization, reduce recurrence, and improve cognitive outcomes for patients. In some embodiments, the patients may have a subdural hematoma. In a preferred embodiment, the intervention is a twist drill, which may be performed at the patient's bedside with decreased anesthesia. In these embodiments, improved efficacy of the twist drill may help decrease perioperative anesthetic complications.

TDC drains placed centrally and anteriorly can result in better drainage results. In addition, placement of the drain site closer to the centroid of a hematoma may not lead to better drainage.

Disclosed herein is a method of optimizing placement of a surgical intervention, to improve results of the intervention and improve patient outcomes. In some embodiments, the method includes
(a) identifying a patient with a lesion that may be treated with a surgical intervention,
(b) imaging a portion of the patient's anatomy to visualize the lesion in three dimensions—for example, by using a CT scan of a patient's head,
(c) identifying the lesion on the image, for example a subdural hematoma,
(d) identifying one or more characteristics of the lesion selected from volume,
   3D centroid,
   anteroposterior axis,
   craniocaudal axis, and
   the center of the hematoma along the craniocaudal axis;
(e) marking an optimal area on the image, the optimal area being positioned near the anterior end of the anteroposterior axis; and the center of the hematoma; wherein
   the optimal area indicates the preferred or ideal location of the surgical intervention for improving the results of the intervention.

In many embodiments, the lesion is a subdural hematoma, the surgical intervention is a twist drill craniostomy, and the result is drainage of the hematoma. In these embodiments, the result may be drainage of 70% or more of the subdural hematoma, for example, greater than about 60%, 65%, 70%, 75%, 80%, 85%, 90%, or 95% and less than about 100%, 95%, 90%, 85%, 80%, 75%, 70%, or 65%.

Imaging of the lesion may be performed by any method known to those of skill in the art, wherein the imaging technique may render a three dimensional image of the lesion. Examples include medical resonance imaging (“MRI”), computed tomography (“CT”), ultrasound (“US”), images from microscopes, or any other device.

### Computer-Aided Program, Application, or System

A computer-aided program or system for optimizing placement of the site of a surgical intervention is described herein. Implementations of the observer matter and the functional operations described herein can be implemented in other types of digital electronic circuitry, or in computer software, firmware, or hardware, including the structures disclosed in this specification and their structural equivalents, or in combinations of one or more of them.

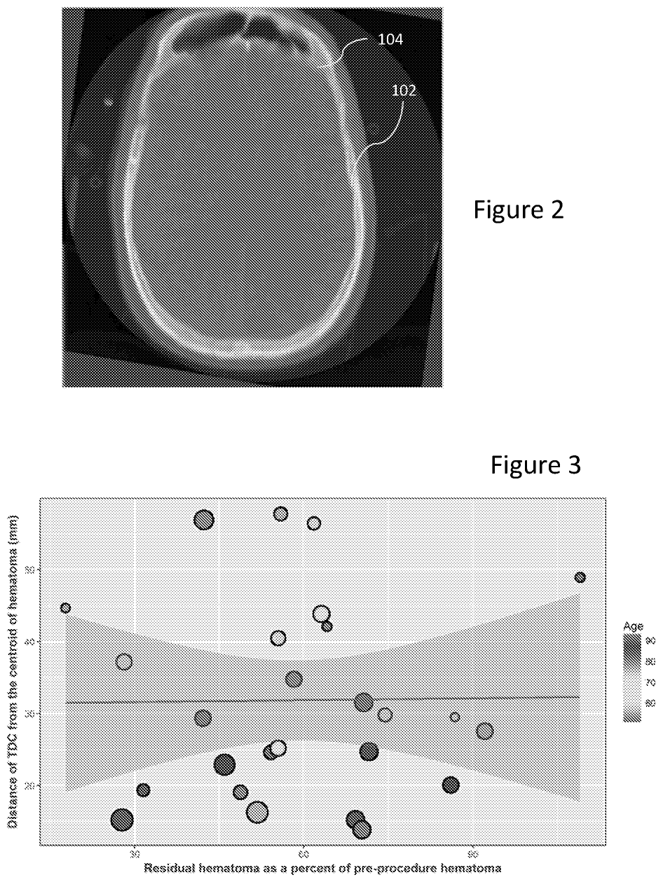

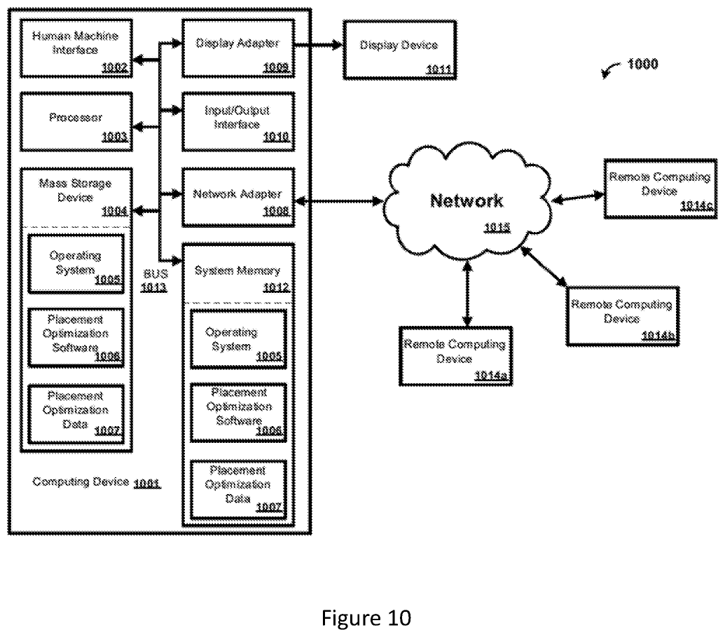

FIG. 10 shows a system 1000 for optimizing placement of the site of a surgical intervention including a computing device 1001 as shown in FIG. 2.

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as placement optimization data 1007 and/or program modules such as operating system 1005 and placement optimization software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards. CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and placement optimization software 1006 may be stored on the mass storage device 1004. One or more of the operating system 1005 and placement optimization software 1006 (or some combination thereof) may comprise program modules and the placement optimization software 1006. Placement optimization data 1007 may also be stored on the mass storage device 1004. Placement optimization data 1007 may be stored in any of one or more databases known in the art. The databases may be centralized or distributed across multiple locations within the network 1015.

A user (e.g., the clinician) may enter commands and information into the computing device 1001 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 1003 via a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 via an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 via Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a,b,c may be made via a network 1015, such as a local area network (LAN) and/or a general wide area network (WAN). Such network connections may be through a network adapter 1008. A network adapter 1008 may be implemented in both wired and wireless environments. Such networking environments are conventional and commonplace in dwellings, offices, enterprise-wide computer networks, intranets, and the Internet.

Application programs and other executable program components such as the operating system 1005 are shown herein as discrete blocks, although it is recognized that such programs and components may reside at various times in different storage components of the computing device 1001, and are executed by the one or more processors 1003 of the computing device 1001. An implementation of placement optimization software 1006 may be stored on or sent across some form of computer readable media. Any of the disclosed methods may be performed by processor-executable instructions embodied on computer readable media.

In some embodiments, the computing device 1001 may be electronically connected to one or more imaging devices, for example a device or system for performing one or more of computed tomography, radiography, medical resonance imaging, or ultrasound.

Optionally, the computing device 1001 can comprise a machine learning module that is operated through the one or more processors 1003 as disclosed herein. The machine learning module can be configured, for example, to use algorithms to build a mathematical model based on experimental data to determine an optimized area in a three dimensional representation of the subject, wherein the optimized area identifies an ideal placement of the surgical intervention site. The machine learning module can make use of any conventional machine learning framework, including, for example and without limitation, a neural network, a decision tree, a support vector machine, and the like. Optionally, in exemplary aspects, the machine learning module can receive data from previous procedures, such as for example, lesion geometry and position within the patient, surgical intervention site (e.g., drain placement location) with respect to the position of the lesion within the patient, and success of the outcome (e.g., residual hematoma volume after draining). The machine learning module can process the data from previous procedures to determine an algorithm for providing a surgical intervention site to optimize success for the pending procedure. The algorithm can optionally use information of a pending procedure (e.g., lesion geometry and position with the patient) to provide the surgical intervention site. Accordingly, the algorithm for determining the surgical intervention site can evolve as more prior surgical data is introduced.

In further embodiments, instead of employing machine learning, the processor can employ a fixed algorithm to determine the optimized surgical intervention site. For example, the processor can be configured to determine a three-dimensional geometrical profile of a lesion using conventional geometric calculations based on one or more images of the lesion. The processor can be further configured to determine the anterior pole and the center of the lesion using conventional geometric and volumetric measurements based upon the previously determined three-dimensional geometrical profile, which can include dimensions of the lesion relative to multiple axes as disclosed herein.

### Examples

### Materials and Methods

Patient selection: A hospital database was searched for twist drill craniostomies (TDC) performed over a 6 year period with digital CT head scans before and after procedure. Scans were excluded if: 1) Procedure was a repeat attempt; 2) SDH was bilateral or 3) Time interval between the scans before and after subdural was more than one week.

CT protocol: All CT scans were performed on a Toshiba Aquilion 16 or Aquilion 64 helical scanner (Toshiba Medical Systems, Tustin, Calif.). Acquisition parameters were as follows: peak tube voltage 120 kVp, x-ray tube current 150-300 mAs, field of view 20-25 cm yielding in-plane resolution 0.412-0.478 mm, soft-tissue reconstruction kernel FC64 or FC67, matrix size 512×512, 27-58 slices, and axial-slice thickness 3-5 mm.

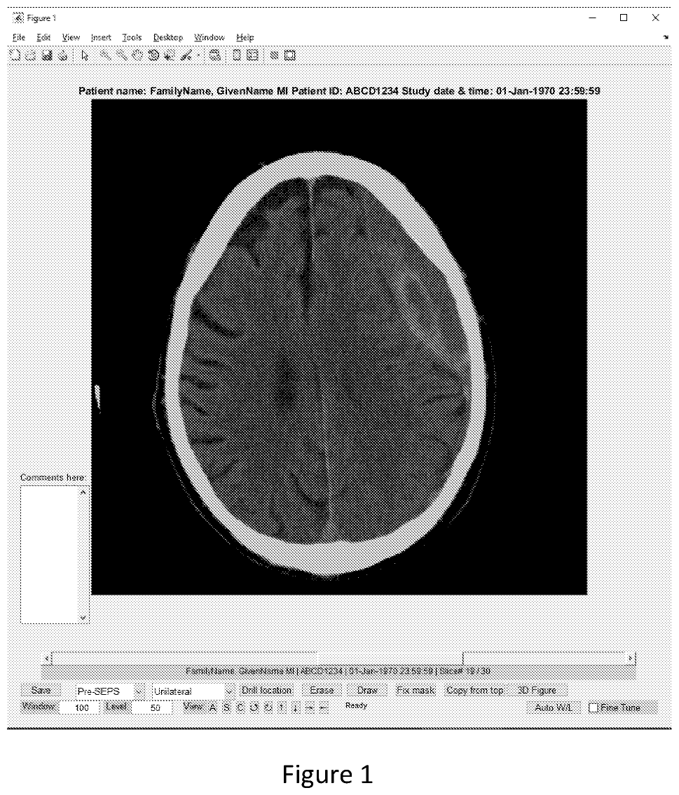

Image analysis technique: The SDH was manually identified by an expert on pre & post procedure CT head scans. The interface of the software used to perform this manual segmentation is shown in FIG. 1. In order to assist the clinician to quickly segment the SDH, a ‘fix mask’ button was provided to eliminate all the areas of the mask that lie outside of the intra-cranial cavity (ICC). The identification of ICC was performed using previously published technique²¹.

On the post procedure scan, the location where TDC was inserted on the inner table of the skull was identified. Once the subdural hematoma segmentation was complete, the clinician made sure that the CT scan is parallel to the figure in axial, coronal, and sagittal views. Next, the clinician saved the SDH mask, along with the transformation used to make CT scan parallel to the figure, by pressing the ‘save’ button shown at the bottom left corner. The hematoma volumes from pre-procedure and post-procedure scans were calculated. As a proxy for the thickest portion of the cSDH, the 3D centroid was calculated for pre-procedure hematoma volume. The centroid was then projected onto the skull to identify the bony location on the inner table of the skull closest to the centroid.

Finally, the pre- and post-procedure CT head scans were coregistered using intensity-based coregistration with the pre-procedure CT head as a fixed target for the post-procedure scan. The coregistration thus obtained was manually inspected for errors and was used only if a good quality coregistration was obtained. As shown in FIG. 2, discussed above, misalignment was one error that was inspected for. In this case, FIG. 2 shows one image, although the entire scan was examined—from the very top of the head to the very bottom of the skull, comprising greater than 20 images to determine this. Dark gray areas 104 in this image indicate poor coregistration, which was considered an error. Conversely, good quality coregistrations had zero to very little dark gray areas 104 surrounding skull and instead have light areas 102 corresponding with good quality coregistration.

The TDC visible on post-procedure scan was then projected onto the pre-procedure scan. The projected drain site was measured as a percent of hematoma length along the y-axis (anteroposterior axis) from zero to one, with zero being on the tip of hematoma and one being on very posterior end of the hematoma and the z-axis (craniocaudal axis), with zero being drain on the caudal-most end of the hematoma and one being drain on the cranial-most end of the hematoma. The projected drain distance from 3D centroid was calculated and expressed in millimeters.

Statistical analysis: All statistical analyses were carried out using Statistical Package for the Social Sciences (SPSS version 24, IBM Corporation, Armonk, N.Y., USA). The residual hematoma volume was expressed as a percent of initial, pre-procedure hematoma volume. Linear regression was preformed to correlate the residual hematoma volume with the distance from the 3D centroid, the anteroposterior axis, and the craniocaudal axis using two tailed significance of 0.05.

### Results

A total of twenty-six patients (all males) of ages 51.9 to 93.9 years were studied. Table 1 shows the descriptive statistics for the cohort studied. FIG. 2 shows result of a typical coregistration of post procedure scan to pre-procedure scan. The following factors were not shown to influence the amount of drainage as confounders (p-values>0.05): Age (Pearson correlation=−0.187), hours between the scans (Pearson correlation=−0.169) and size of the hematoma prior to drainage (Pearson correlation=−0.216). Therefore, these parameters were not included in any of the models built.

TABLE 1
Descriptive statistics

| Parameter | Mean | Median | Std. Deviation |
|---|---|---|---|
| Age (years ) | 76.75 | 80.50 | 10.52 |
| Hours between scans | 30.83 | 25.24 | 27.92 |
| Pre-Volume (ml) | 131.64 | 138.22 | 52.18 |
| Post-Volume (ml) | 75.36 | 72.03 | 34.20 |
| Absolute Decrease (ml) | 56.28 | 52.65 | 37.63 |
| Post-Vol. (percent of pre) | 59.00 | 57.06 | 21.21 |

The drain position was, on average, 32.63 mm (S.D.=16.26, min=7.98, max=67.45) away from the centroid of the hematoma. However, the distance of drain from the centroid of the hematoma did not correlate with the amount of hematoma drained (R=0.014, p=0.947, see FIG. 3 and Table 2).

TABLE 2
Association of residual hematoma as a percent of initial hematoma versus distance
of twist drill craniostomy from the centroid of hematoma using linear regression.

| | Unstandardized | | | | | 95.0% Confidence | |
|---|---|---|---|---|---|---|---|
| | Coefficients | | Standardized | | | Interval for B | |
| | | Std. | Coefficients | | | Lower | Upper |
| Model | B | Error | Beta | t | Sig. | Bound | Bound |
| (Constant) | 58.314 | 11.065 | | 5.27 | 0 | 35.476 | 81.152 |
| Distance of TDC from the | 0.021 | 0.321 | 0.014 | 0.067 | 0.947 | −0.641 | 0.684 |
| centroid of hematoma | | | | | | | |

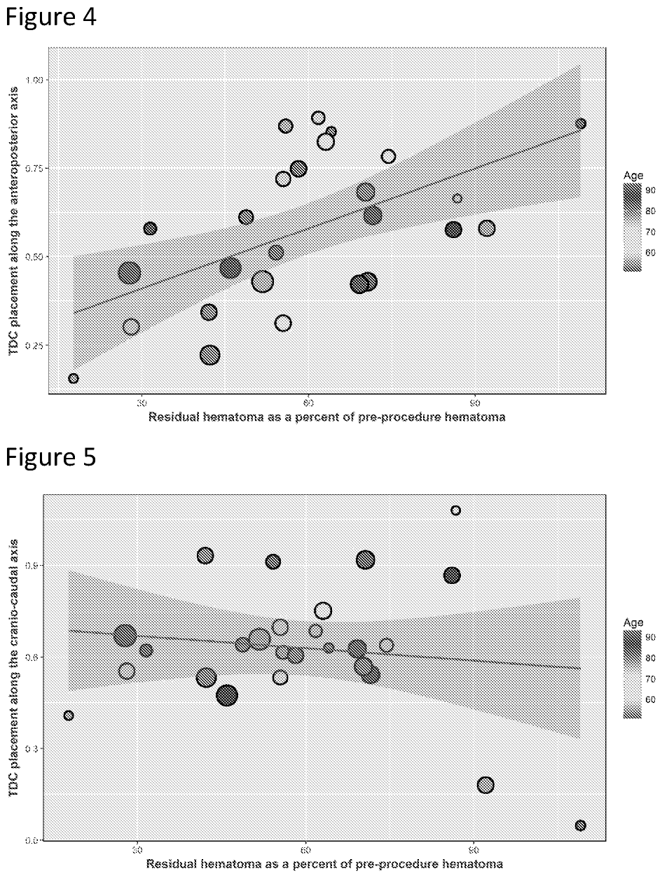

The drain was on average placed at 57.42% (S.D.=21.26%, min=15.58%, max=89.28%) along the anteroposterior axis of the hematoma, where 0% would mean a drain at the very anterior pole of the hematoma while 100% would mean a drain at the very posterior pole of the hematoma. As described herein, it is contemplated that the “anterior pole” can refer to the most anterior portion of a lesion (measured relative to an anteroposterior axis). The drain location along the anteroposterior axis was strongly correlated with the percentage of hematoma left as residual after treatment (R=0.566, p-value=0.003, see Table 3 and FIG. 4).

TABLE 3
Association of residual hematoma as a percent of initial
hematoma versus the placement of twist drill craniostomy
along anteroposterior (AP) axis using linear regression.

| | Unstandardized | | | | | 95.0% Confidence | |
|---|---|---|---|---|---|---|---|
| | Coefficients | | Standardized | | | Interval for B | |
| | | Std. | Coefficients | | | Lower | Upper |
| Model | B | Error | Beta | t | Sig. | Bound | Bound |
| (Constant) | 26.596 | 10.257 | | 2.593 | 0.016 | 5.426 | 47.766 |
| TDC Placement | 56.430 | 16.791 | 0.566 | 3.361 | 0.003 | 21.775 | 95.086 |
| along AP axis | | | | | | | |

Along the craniocaudal axis, the drains were placed at a mean of 63.03% (S.D.=21.82%, min=4.79%, max=107.99%), where 0% should be understood to mean that a drain is located at the very bottom (caudal) end of the hematoma, and 100% should be understood to mean that a drain is located at the very top (cranial) end of the hematoma. The drain location along the craniocaudal axis was not correlated with the residual percentage of hematoma left inside cranial cavity after treatment (R=0.132, p-value=0.522, see FIG. 5 and Table 4).

TABLE 4
Association of residual hematoma as a percent of initial hematoma versus the placement
of twist drill craniostomy along craniocaudal axis (z-axis) using linear regression.

| | Unstandardized | | | | | 95.0% Confidence | |
|---|---|---|---|---|---|---|---|
| | Coefficients | | Standardized | | | Interval for B | |
| | | Std. | Coefficients | | | Lower | Upper |
| Model | B | Error | Beta | t | Sig. | Bound | Bound |
| (Constant) | 67.057 | 13.095 | | 5.121 | 0.000 | 40.031 | 94.083 |
| TDC Placement | −12.786 | 19.672 | −0.132 | −0.650 | 0.522 | −53.388 | 27.816 |
| along z-axis | | | | | | | |

An analysis of z-axis placement was performed to investigate a correlation between how far away the drain was from the center of the hematoma along z-axis with the residual hematoma. The distance from center was defined as:

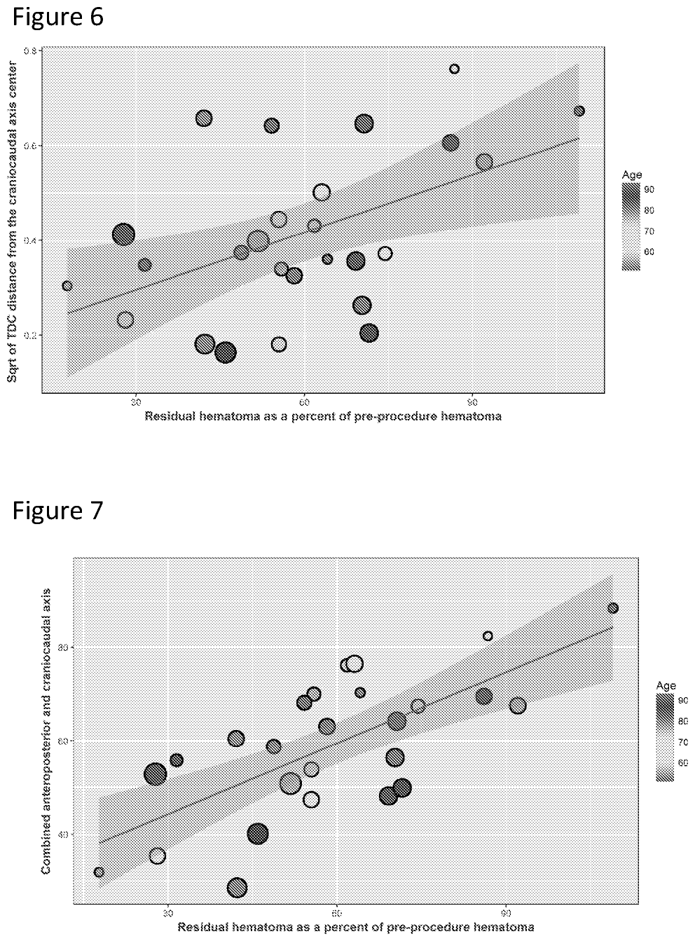

z′=|0.5−z|

Where z is the original distance along the z-axis from bottom to top as a fraction of hematoma height and z′ is the resulting distance calculated from the center. The drains were on average placed at 19.9% (S.D.=15.5%, min=2.7%, max=57.9%) away from the center of the hematoma, where 0% should be understood to mean that a drain is located at the center of the hematoma, while 50% should be understood to mean that the drain is located at the very caudal or cranial end of the hematoma. The z′ was not normally distributed. Hence, when used in modeling, it was converted into normal distribution by taking a square root, \hat{z}. The analysis was significant for the correlation of placement towards the center of the hematoma and the residual hematoma percent (R=0.502, p-value=0.009, see FIG. 6 and Table 5).

TABLE 5
Association of residual hematoma as a percent of initial hematoma versus
the placement of twist drill craniostomy towards the center of the hematoma
along craniocaudal axis (z-axis) using linear regression.

| | Unstandardized | | | | | 95.0% Confidence | |
|---|---|---|---|---|---|---|---|
| | Coefficients | | Standardized | | | Interval for B | |
| | | Std. | Coefficients | | | Lower | Upper |
| Model | B | Error | Beta | t | Sig. | Bound | Bound |
| (Constant) | 33.301 | 9.754 | | 3.414 | 0.002 | 13.17 | 53.432 |
| Sqrt of TDC distance | 62.198 | 21.872 | 0.502 | 2.844 | 0.009 | 17.057 | 107.34 |
| from center as a | | | | | | | |
| fraction of hematoma (\hat{z}) | | | | | | | |

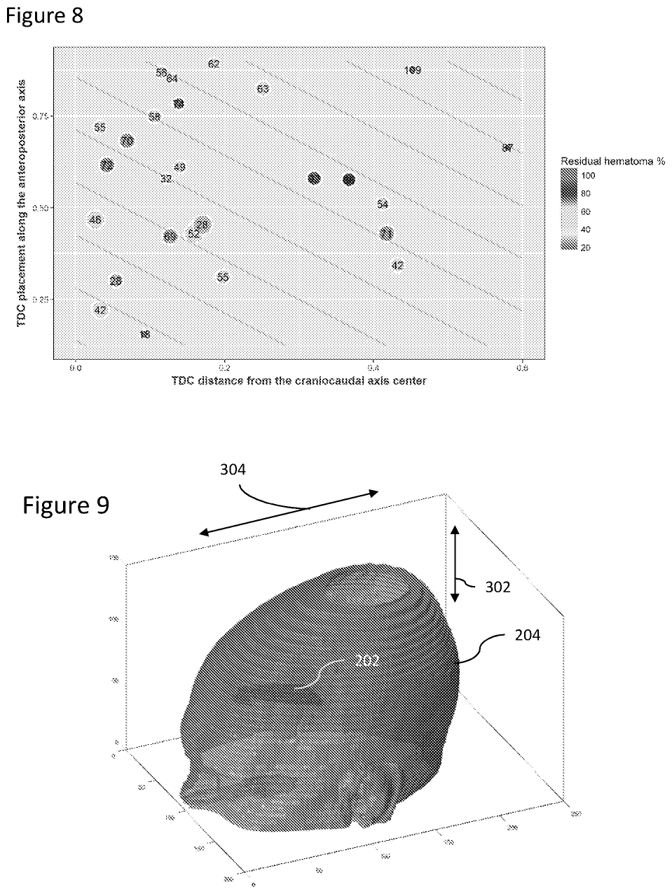

In order to find if the correlation of residual hematoma with the placement towards the center along the z-axis was due to confounding from the relationship of residual hematoma with anteroposterior axis, tests were performed to determine if \hat{z} was correlated to distance along y-axis. Pearson correlation revealed both variables to be independent of each other (R=0.130, p-value=0.526). Since both variables were independent of each other, we combined both of them into one model. The final model with distance along y-axis and \hat{z} had R=0.712 with a p-value<0.001, see Table 6, FIG. 7, and FIG. 8.

TABLE 6
Association of residual hematoma as a percent of initial hematoma versus the placement
of twist drill craniostomy along anteroposterior axis and towards the center
of the hematoma along craniocaudal axis (z-axis) using linear regression.

| | Unstandardized | | | | | 95.0% Confidence | |
|---|---|---|---|---|---|---|---|
| | Coefficients | | Standardized | | | Interval for B | |
| | | Std. | Coefficients | | | Lower | Upper |
| Model | B | Error | Beta | t | Sig. | Bound | Bound |
| (Constant) | 7.548 | 11.017 | | 0.685 | 0.5 | −15.242 | 30.338 |
| TDC Placement | 50.764 | 14.736 | 0.509 | 3.445 | 0.002 | 20.28 | 81.249 |
| along AP axis | | | | | | | |
| Square roof of TDC | 53.979 | 18.303 | 0.436 | 2.949 | 0.007 | 16.117 | 91.841 |
| distance from center | | | | | | | |
| as a fraction of SDH (\hat{z}) | | | | | | | |

Finally, since the anterior and central placement of the drains are so strongly associated with the decreased amount of the residual hematoma, the software is configured to show that location on the CT scan. In FIG. 1, clicking the “Drill location” button can display an optimal area, drawn along the area where the residual hematoma is predicted to be 20% or less. In some embodiments, the optimal (or “golden”) area may be projected onto the patient's body to aid in locating the intervention site (here, a drill location). Referring to FIGS. 1 and 9, clicking the button “3D Figure” of the user interface can cause the computing device to display the optimal area 202 on a 3D FIG. 204.

In embodiments wherein the optimal area is projected onto the patient's body, coregistration may be used. In this embodiment, coregistration may include using information from the image (or images) of interest and the surrounding environment. The mutual information is then used to place the image of interest (for example the golden or optimal area) relative to the environment. In this embodiment, for example, coregistration can be accomplished in multiple ways, some of which are: 1) use of a holographic rendering of patient's skin visible on medical image of interest and correlate that with the actual skin sensed by the AR system; 2) use the skin as described above, except for the fact that the user (for example a technician or physician) manually adjusts the holographic rendering of the skin relative to the patient's body; 3) use of additional fiducials placed on patient's body that are a) visible on medical image b) can be sensed by an augmented reality (AR) system; and 4) any other method of 3D scanning can be used as a sensor in AR system, and the resulting information can be correlated with the image of interest, in turn enabling the accurate placement of the holograms relative to the patient body.

### DISCUSSION

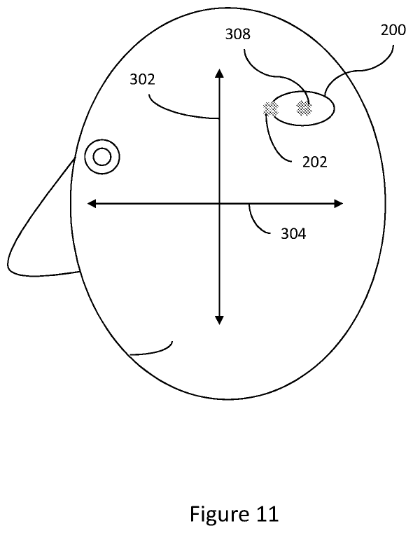

A simple linear model can be used to develop an algorithm and computer imaging program for optimization of twist drill craniostomy drain placement to treat cSDH. Referring to FIG. 11, it can be shown that, compared to a drain placed at the very posterior end of a hematoma 200 along the anteroposterior axis 304, a drain placed at the very anterior pole decreases the size of residual hematoma by 56.6%. Additionally, placing drain(s) at the very middle of the hematoma along the craniocaudal axis (z-axis) 302 can be associated with 50% more drainage. Surprisingly, it can be shown that these two factors may be combined to significantly enhance drainage (the combination of factors accounts for 71% of the total drainage of the SDH).

Previously, it was thought that placing the drain at the point of maximum thickness, or the centroid 308, would best drain the hematoma. However, it is shown herein that this practice does not typically get the best results. The 3D centroid was used as a proxy for the thickest portion of the hematoma and all measurements of distances between the actual and hypothetical sites were performed on the inner table of the skull. Hence, the methods disclosed herein for measurement of 3D hematoma are superior to previous methods, as the disclosed methods tends to be resistant to irregularly shaped hematomas.

In conjunction with model building, tests were conducted to determine if the effect observed was solely due to confounders including age, size of the hematomas—with bigger hematomas allowing anterior drainage and better results or the number of hours between the scans—with longer intervals hypothetically leading to more drainage. However, as shown in FIG. 4, none of these parameters were found to explain drainage better than a random variable (p-values>0.05). Hence, Applicants have shown that a central anterior placement of the drain can enhance drainage.

That the anterior placement of the drains can lower the residual hematoma volume initially appears to be counterintuitive. One of skill in the art may reasonably believe that gravity will pull the blood down allowing for better drainage in posteriorly placed drains, but the data clearly suggests the contrary is true. One reason for this outcome may be the fact that since humans lay down on their backs, the posterior part of the hematoma has a higher tendency to organize and become fibrotic or resistant to drainage. Another reason may be the fact that during surgery, when two burr holes are drilled to drain SDH, the brain is commonly seen expanding back in the posterior hole prior to the anterior burr hole. This may lead to, in the case of TDC, a blockage of the TDC drain by the brain, leading to decreased drainage.

The presently disclosed model is parsimonious, using only the length of the hematoma along the anteroposterior axis and the distance from the center of the z-axis. None of the other factors disclosed herein affected the hematoma draining, so they are excluded from the model. The disclosed method, therefore, allows for better conceptualization and lowers the risk of overfitting to the data and arriving at erroneously high accuracy. Use of the disclosed method can substantially reduce the TDC failure rate.

Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A method for optimizing placement of a surgical intervention site for a surgical intervention in a human or animal subject, the method comprising: imaging a lesion in the subject; segmenting the lesion; identifying a center of the lesion along a z-axis; identifying an anterior pole of the lesion along an anteroposterior axis; and displaying a location for the surgical intervention in a three dimensional representation of at least a portion of the subject.

Aspect 2: The method as in aspect 1, wherein imaging the lesion in the subject comprises using an imaging method selected from radiography, computed tomography, medical resonance imaging, or ultrasound.

Aspect 3: The method as in aspect 1 or aspect 2, wherein at least a portion of the method is performed by a processor executing a computer program.

Aspect 4: The method as in aspect 3, wherein the processor, when executing the computer program, applies a machine learning algorithm for determining the location for the surgical intervention.

Aspect 5: The method as in aspect 2 or aspect 3, wherein the processor, when executing the computer program, provides, on a visual output, an interface for displaying and coregistering a pre-procedure image and a post-procedure image.

Aspect 6: The method as in aspect 5, wherein coregistering is performed using an intensity based coregistration, wherein the pre-procedure image is a fixed target.

Aspect 7: The method as in any of aspects 1-6, further comprising performing a surgical intervention, wherein the surgical intervention is an incision, a drainage, a drilling, or a combination thereof.

Aspect 8: The method as in any of aspects 1-7, wherein the surgical intervention site is a drill site.

Aspect 9: The method as in any of aspects 1-8, wherein the lesion is a collection or accumulation of fluid within a brain, a spine, a subdural space, or an epidural space of the subject.

Aspect 10: The method as in any of aspects 1-9, wherein the lesion is a subdural hematoma, wherein performing the surgical intervention comprises draining more than about 70% of a volume of the subdural hematoma.

Aspect 11: The method as in aspect 10, wherein performing the surgical intervention comprises draining more than about 80% of the volume of the subdural hematoma.

Aspect 12: The method as in any of aspects 1-11, wherein the location for the surgical intervention site is approximately at the anterior pole of the lesion along the anteroposterior axis and approximately at the center of the lesion along the z-axis.

Aspect 13: A method for assessing the volumetric distribution of a brain lesion, comprising: imaging the brain lesion; using a processor, performing segmentation analysis of an image of the brain lesion to determine an anterior pole of the brain lesion along an anteroposterior axis and a center of the brain lesion along a z-axis; using the processor, creating a model of the brain lesion including the anterior pole of the brain lesion and the center of the brain lesion along the z-axis; and using the processor, identifying the anterior pole of the brain lesion along the anteroposterior axis and the center of the brain lesion along the z-axis as a location for a surgical approach to treat the brain lesion.

Aspect 14: The method of aspect 13 wherein the brain lesion is a collection or accumulation of fluid within a brain, a spine, a subdural space, or an epidural space.

Aspect 15: The method of claim 13 or claim 14, wherein the surgical approach is a twist drill craniostomy.

Aspect 16: The method of any of aspects 13-15 wherein the surgical approach comprises placing a drain for a subdural hematoma.

Aspect 17: The method of any of aspects 13-16, wherein identifying the anterior pole of the brain lesion and the center of the brain lesion along the z-axis as the location for the surgical approach comprises locating a treatment site for a treatment, wherein the treatment is one of a surgical incision, a cranial drill site, a craniostomy location, a craniotomy location, and a craniectomy location.

Aspect 18: The method of any of aspects 13-17, wherein the segmentation analysis includes an analysis of images to distinguish between brain tissue and non-brain space.

Aspect 19: The method of claim 18, wherein the non-brain space is an intracranial space containing one or more of cerebrospinal fluid, air, blood, a tumor, an abscess, a nodule, and an inflammatory lesion.

Aspect 20: The method any of aspects 13-19, wherein the segmentation analysis includes an analysis of one or more of a density of the brain lesion, volume of the brain lesion, area of distribution of the brain lesion, and a gravitational force acting upon the brain lesion.

Aspect 21: The method of aspect 17, wherein identifying the anterior pole of the brain lesion and the center of the brain lesion along the z-axis as the location for the surgical approach comprises using the processor to analyze and compare pre-procedure and post-procedure imaging from patients who have undergone the treatment.

Aspect 22: The method of aspect 13, further comprising performing the surgical approach at the identified location.

Aspect 23: A method for assessing the volumetric distribution of a brain lesion, comprising:

imaging the brain lesion;

using segmentation analysis to analyze the brain lesion;

creating a model of the brain lesion; and

identifying a location for a surgical approach to treat the brain lesion.

Although the foregoing invention has been described in some detail byway of illustration and example for purposes of clarity of understanding, certain changes and modifications may be practiced within the scope of the appended claims.

### REFERENCES

The following references are hereby incorporated by reference herein for all purposes:
1. Balser, D., Farooq, S., Mehmood, T., Reyes, M. & Samadani, U. Actual and projected incidence rates for chronic subdural hematomas in United States Veterans Administration and civilian populations. J Neurosurg 123, 1-7, doi:10.3171/2014.9.jns141550 (2015).
2. Miranda, L. B., Braxton. E., Hobbs, J. & Quigley, M. R. Chronic subdural hematoma in the elderly: not a benign disease. J Neurosurg 114, 72-76, doi:10.3171/2010.8.jns10298 (2011).
3. Frati, A. et al. Inflammation markers and risk factors for recurrence in 35 patients with a posttraumatic chronic subdural hematoma: a prospective study. J Neurosurg 100, 24-32, doi:10.3171/Jns.2004.100.1.0024 (2004).
4. Ohba. S., Kinoshita, Y., Nakagawa, T. & Murakami, H. The risk factors for recurrence of chronic subdural hematoma. Neurosurgical review 36, 145-149; discussion 149-150, doi:10.1007/s10143-012-0396-z (2013).
5. Almenawer, S. A. et al. Chronic subdural hematoma management: a systematic review and meta-analysis of 34,829 patients. Annals of surgery 259, 449-457, doi:10.1097/sla.0000000000000255 (2014).
6. Liu, W., Bakker, N. A. & Groen, R. J. Chronic subdural hematoma: a systematic review and meta-analysis of surgical procedures. J Neurosurg 121, 665-673, doi:10.3171/2014.5.jns132715 (2014).
7. Balser, D. et al. Evolving management of symptomatic chronic subdural hematoma: experience of a single institution and review of the literature. Neurol Res 35, 233-242, doi:10.1179/1743132813Y.0000000166 (2013).
8. Safain, M. et al. A single center's experience with the bedside subdural evacuating port system: a useful alternative to traditional methods for chronic subdural hematoma evacuation. J Neurosurg 118, 694-700, doi:10.3171/2012.11.JNS12689 (2013).
9. Frontera, J. A. et al. Trend in outcome and financial impact of subdural hemorrhage. Neurocritical care 14, 260-266, doi:10.1007/s12028-010-9418-2 (2011).
10. Hamilton, M. G., Frizzell, J. B. & Tranmer, B. I. Chronic subdural hematoma: the role for craniotomy reevaluated. Neurosurgery 33, 67-72 (1993).
11. De Jesus, O., Pacheco, H. & Negron, B. Chronic and subacute subdural hematoma in the adult population. The Puerto Rico experience. P R Health Sci J 17, 227-233 (1998).
12. Iantosca, M. R. & Simon, R. H. Chronic subdural hematoma in adult and elderly patients. Neurosurg Clin N Am 11, 447-454 (2000).
13. Altamura, S. & Muckenthaler, M. U. Iron toxicity in diseases of aging: Alzheimer's disease, Parkinson's disease and atherosclerosis. Journal of Alzheimer's disease: JAD 16, 879-895, doi:10.3233/jad-2009-1010 (2009).
14. Hua, Y., Keep, R. F., Hoff, J. T. & Xi, G. Brain injury after intracerebral hemorrhage: the role of thrombin and iron. Stroke 38, 759-762, doi:10.1161/01.str.0000247868.97078.10 (2007).
15. Mills, E., Dong, X. P., Wang, F. & Xu, H. Mechanisms of brain iron transport: insight into neurodegeneration and CNS disorders. Future medicinal chemistry 2, 51-64 (2010).
16. Xi, G., Keep, R. F. & Hoff, J. T. Mechanisms of brain injury after intracerebral haemorrhage. The Lancet. Neurology 5, 53-63, doi:10.1016/s1474-4422(05)70283-0 (2006).
17. Dumont, T. M., Rughani, A. I., Goeckes, T. & Tranmer, B. I. Chronic subdural hematoma: a sentinel health event. World neurosurgery 80, 889-892, doi:10.1016/j.wneu.2012.06.026 (2013).
18. Nagata, K., Asano, T., Basugi, N., Tango, T. & Takakura, K. [Studies on the operative factors affecting the reduction of chronic subdural hematoma, with special reference to the residual air in the hematoma cavity]. No Shinkei Geka 17, 15-20 (1989).
19. Nakaguchi, H., Tanishima, T. & Yoshimasu, N. Relationship between drainage catheter location and postoperative recurrence of chronic subdural hematoma after burr-hole irrigation and closed-system drainage. J Neurosurg 93, 791-795. doi:10.3171/jns.2000.93.5.0791 (2000).
20. Leroy, H. A. et al. Predictors of functional outcomes and recurrence of chronic subdural hematomas. Journal of clinical neuroscience: official journal of the Neurosurgical Society of Australasia 22, 1895-1900, doi:10.1016/j.jocn.2015.03.064 (2015).
21. Bin Zahid, A. et al. Accelerated Brain Atrophy on Serial Computed Tomography: Potential Marker of the Progression of Alzheimer Disease. J Comput Assist Tomogr 40. 827-832, doi:10.1097/RCT.0000000000000435 (2016).

1. A method for optimizing placement of a surgical intervention site for a surgical intervention in a human or animal subject, the method comprising:
imaging a lesion in the subject;
segmenting the lesion;
identifying a center of the lesion along a z-axis;
identifying an anterior pole of the lesion along an anteroposterior axis; and
displaying a location for the surgical intervention in a three dimensional representation of at least a portion of the subject.

2. The method as in claim 1, wherein imaging the lesion in the subject comprises using an imaging method selected from radiography, computed tomography, medical resonance imaging, or ultrasound.

3. The method as in claim 1, wherein at least a portion of the method is performed by a processor executing a computer program.

4. The method as in claim 3, wherein the processor, when executing the computer program, applies an algorithm for determining the location for the surgical intervention.

5. The method as in claim 3, wherein the processor, when executing the computer program, provides, on a visual output, an interface for displaying and coregistering a pre-procedure image and a post-procedure image.

6. The method as in claim 5, wherein coregistering is performed using an intensity based coregistration, wherein the pre-procedure image is a fixed target.

7. The method as in claim 1, further comprising performing the surgical intervention, wherein the surgical intervention is an incision, a drainage, a drilling, or a combination thereof.

8. The method as in claim 1, wherein the surgical intervention site is a drill site.

9. The method as in claim 7, wherein the lesion is a collection or accumulation of fluid within a brain, a spine, a subdural space, or an epidural space of the subject.

10. The method as in claim 9, wherein the lesion is a subdural hematoma, wherein performing the surgical intervention comprises draining more than about 70% of a volume of the subdural hematoma.

11. The method as in claim 10, wherein performing the surgical intervention comprises draining more than about 80/6 of the volume of the subdural hematoma.

12. The method as in claim 1, wherein the location for the surgical intervention site is approximately at the anterior pole of the lesion along the anteroposterior axis and approximately at the center of the lesion along the z-axis.

13. A method for assessing the volumetric distribution of a brain lesion, comprising:
imaging the brain lesion;
using a processor, performing segmentation analysis of an image of the brain lesion to determine an anterior pole of the brain lesion along an anteroposterior axis and a center of the brain lesion along a z-axis;
using the processor, creating a model of the brain lesion including the anterior pole of the brain lesion and the center of the brain lesion along the z-axis; and
using the processor, identifying the anterior pole of the brain lesion along the anteroposterior axis and the center of the brain lesion along the z-axis as a location for a surgical approach to treat the brain lesion.

14. The method of claim 13 wherein the brain lesion is a collection or accumulation of fluid within a brain, a spine, a subdural space, or an epidural space.

15. The method of claim 13, wherein the surgical approach is a twist drill craniostomy.

16. The method of claim 13, wherein the surgical approach comprises placing a drain for a subdural hematoma.

17. The method of claim 13, wherein identifying the anterior pole of the brain lesion and the center of the brain lesion along the z-axis as the location for the surgical approach comprises locating a treatment site for a treatment, wherein the treatment is one of a surgical incision, a cranial drill site, a craniostomy location, a craniotomy location, and a craniectomy location.

18. The method of claim 13, wherein the segmentation analysis includes an analysis of images to distinguish between brain tissue and non-brain space.

19. The method of claim 18, wherein the non-brain space is an intracranial space containing one or more of cerebrospinal fluid, air, blood, a tumor, an abscess, a nodule, and an inflammatory lesion.

20. The method claim 13, wherein the segmentation analysis includes an analysis of one or more of a density of the brain lesion, volume of the brain lesion, area of distribution of the brain lesion, and a gravitational force acting upon the brain lesion.

21. The method of claim 17, wherein identifying the anterior pole of the brain lesion and the center of the brain lesion along the z-axis as the location for the surgical approach comprises using the processor to analyze and compare pre-procedure and post-procedure imaging from patients who have undergone the treatment.

22. The method of claim 13, further comprising performing the surgical approach at the identified location.

23. A method for assessing the volumetric distribution of a brain lesion, comprising:
imaging the brain lesion,
using segmentation analysis to analyze the brain lesion;
creating a model of the brain lesion; and
identifying a location for a surgical approach to treat the brain lesion.
